SEQUENCE LISTING (2 sequences)

SEQ ID NO: 1 - DNA, 3739 bp, Homo sapiens
  misc_feature (0)...(0): Adult Human Aorta cDNA Library
gcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgagagcaatcacctcacagacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaatatggtttccagagaaaaggccaatacctaagcattttttccatgagaagcactgcatacttacctatgtggactataataacctgtctccaaaaccatgccataataatataagtgctttagaaattaaatcattgtgttttttatgcattttgctgaggcatgcttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaattaggtgggatatttgataggatccatatctaataatggattcgaactctccaaactacaccaattaatttaatgtatcttgcttttgtgttcccgtctttttgaaatatagacatggatttataatggcattttatatttggcaggccatcatagattatttacaacctaaaagcttttgtgtatcaaaaaaatcacattttattaatgtaaatttctaatcgtatacttgctcactgttctgatttcctgtttctgaaccaagtaaaatcagtcctagaggctatggttcttaatctatggagcttgctttaagaagccagttgtcaattgtggtaacacaagtttggccctgctgtcctactgtttaatagaaaactgttttacattggttaatggtatttagagtaattttttctctctgcctcctttgtgtctgttttaaaggagactaactccaggagtaggaaatgattcatcatcctccaaagcaagaggcttaagagagaaacaccgaaattcagatagctcagggactgctaacagagaactacattttttcttattgccttgaaagttaaaaggaaagcagatttcttcagtgactttgtggtcctactaactacaaccagtttgggtgacagggctggtaaagtcccagtgttagatgagtgacctaaatatacttagatttctaagtatggtgctctcaggtccaagttcaactattcttaagcagtgcaattcttcccagttatttgagatgaaagatctctgcttattgaagatgtaccttctaaaactttcctaaaagtgtctgatgtttttactcaagaggggagtggtaaaattaaatactctattgttcaattctctaaaatcccagaacacaatcagaaatagctcaggcagacactaataattaagaacgctcttcctcttcataactgctttgcaagtttcctgtgaaaacatcagtttcctgtaccaaagtcaaaatgaacgttacatcactctaacctgaacagctcacaatgtagctgtaaatataaaaaatgagagtgttctacccagttttcaataaaccttccaggctgcaataaccagcaaggttttcagttaaagccctatctgcactttttatttattagctgaaatgtaagcaggcatattcactcacttttctttgcctttcctgagagttttattaaaacttctcccttggttacctgttatcttttgcacttctaacatgtagccaataaatctatttgatagccatcaaaggaataaaaagctggccgtacaaattacatttcaaaacaaaccctaataaatccacatttccgcatggctcattcacctggaataatgccttttattgaatatgttcttatagggcaaaacactttcataagtagagttttttatgttttttgtcatatcggtaacatgcagctttttcctctcatagcattttctatagcgaatgtaatatgcctcttatcttcatgaaaaataaatattgcttttgaacaaaaaaaaaaaaaaaaaa

SEQ ID NO: 2 - protein, 370 aa, Homo sapiens
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

(19) United States
(12) Patent Application Publication
(10) Pub. No.: US 2004/0110683 A1
(43) Pub. Date: Jun. 10, 2004

(54) Cell proliferation factor Fwa267

(75) Inventors: Rutai Hui, Beijing (CN); Jingzhou Chen, Beijing (CN); Baohua Liu, Beijing (CN); Yuqing Liu, Beijing (CN)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Cardiovascular Institute of Chinese Academy of Medical Sciences

(21) Appl. No.: 10/650,284

(22) Filed: Aug. 28, 2003

Related U.S. Application Data
(63) Continuation of application No. PCT/CN02/00126, filed on Feb. 28, 2002.

(30) Foreign Application Priority Data
Feb. 28, 2001 (CN) 01109260.2

(51) Int. Cl. A61K 38/18; C07K 14/475; C07H 21/04

(52) U.S. Cl. 514/12; 530/399; 435/69.1; 435/320.1; 435/325; 536/23.5

(57) ABSTRACT

The application disclosed a cell proliferation factor-Fwa267 polypeptide and the polynucleotide encoding the polypeptide and a process for producing the polypeptide by recombinant method, the antibody and antagonist against the polypeptide and a composition comprising Fwa267 polypeptide and its usage in the treatment of angiocardiopathy and cancer are also disclosed. The application further disclosed a diagnostic method by detecting the mutation in the nucleic acid sequence encoding Fwa 267 or the amount of Fwa267 in the sample or the changes of autoantibody of Fwa267 gene product. The cell proliferation factor of the present application is of human resource.

### CROSS-REFERENCE TO RELATED APPLICATIONS

This application is a continuation of International Patent Application PCT/CN02/00126 filed Feb. 28, 2002, which designated the United States and is hereby incorporated herein in its entirety by reference, and claims the benefit of Chinese Application No. 01109260.2 filed Feb. 28, 2001.

### FIELD OF THE INVENTION

The present invention relates to the most recently identified polynucleotides and the polypeptides encoded by said polynucleotides, methods for producing said polynucleotides and polypeptides, and their various uses. The polypeptides of the present invention have been identified as cell proliferation factors, also referred to as “Fwa267” below. The polynucleotides and polypeptides of the present invention come from humans.

### BACKGROUND OF THE INVENTION

Cardio-cerebral-vascular diseases are among the greatest threats to human health. Statistics indicate that 2.6 million of our compatriots in China die of these diseases every year, an average of one person every 12 seconds. Nationally, the death-rate from these diseases as a percentage of total deaths has increased from 12.1% in 1957 to 35.8% in 1990, an increase of 2.9 times. According to predictions made by the World Bank, the death-rate worldwide from cardio-cerebral-vascular diseases as a percentage of total deaths will increase from 28.9% in 1990 to 36.3% in the year 2020, and 70% of those deaths will occur in developing nations. There are currently more than 100 million people in China suffering from high blood pressure, and that number is increasing at a rate of 3.5 million per year; those incapacitated by cerebral strokes number six million, increasing by 1.5 million per year; those with coronary heart disease number one million, increasing by 500 thousand per year; and cardiomyopathy patients number three million. Preventing cardio-cerebral-vascular diseases is an extremely important means of both lightening the resulting economic load on humanity and ensuring good health.

The treatment of cardio-cerebral-vascular diseases has progressed through four major stages. During the 1920s research in circulation dynamics revealed that the heart was a “pump,” establishing a foundation for the treatment of heart failure. Then, in the 1960s and '70s knowledge of and methods for handling dangerous factors in cardiovascular diseases reduced the heart attack rate for people with heart disease in western nations to close to 40%. In 1970, research in cardiac cellular electrophysiology resulted in new testing methods and treatments for irregular heart rhythms. By the late '80s/early 90's knowledge in the field of vascular biology resulted in new methods of coronary intervention.

Treatments for heart failure, antiarrhythmic drugs and intervention are now used extensively to improve the quality of life for patients suffering from cardio-cerebral-vascular diseases. Nevertheless, they treat only the symptoms, and do not reach the cause of the diseases at all. While extending the lives of patients, they do not truly achieve the goals of prevention and cure. Therefore, research was undertaken at the level of molecular biology to ascertain the factors leading to cardio-cerebral-vascular diseases and the mechanism behind attacks of this kind with the hope of obtaining breakthroughs in treating diseases at their root.

An important research tool in the realm of biological engineering is the cDNA library. Typically, mRNA is extracted from cells and reverse transcriptase is used to synthesize a DNA replica (cDNA: “Complementary DNA”). Single-stranded cDNA molecules in DNA polymerase are transformed into double-stranded DNA molecules, and then added to vectors and transferred to host bacteria to grow clones. Each clone, then, contains only certain mRNA information. A set of clones is called a cDNA library.

Because cDNA does not include introns, expressed genes can be directly screened from out of the cDNA library. In contrast to the gene pool, the cDNA library has the advantages of simplicity and convenience. The divergence between expressed genes of different human cells determines the divergence between the phenotypes of their tissue and organs. An effective method of studying genetic cardio-cerebral-vascular diseases is to isolate and identify distinctly-expressed genes from the aortic cDNA library, especially to isolate and identify the causes of the diseases.

### SUMMARY OF THE INVENTION

Based upon one aspect of the present invention, what is provided is a new type of mature polypeptide Fwa267, along with biologically active Fwa267 fragments, analogs and derivatives useful for diagnosis or treatment. The polypeptide of the present invention comes from humans.

Based upon another aspect of the present invention, what is provided are isolated nucleic acid molecules encoding the polypeptides of the present invention, including mRNA, DNA, cDNA, genome DNA and biologically active nucleic acid fragments, analogs and derivatives useful in diagnosis and treatment.

Based upon another aspect of the present invention, what is provided are methods for producing Fwa267 using recombinant techniques. Said methods include cultivating the recombinant prokaryotic and/or eukaryotic host cells of the nucleic acid sequences which encode the polypeptides of the present invention.

Based upon another aspect of the present invention, what is provided is a treatment method using Fwa267 polypeptide or the polynucleotide which encodes Fwa267 polypeptide, such as the inhibition of the formation of tumors in the treatment of cardiovascular proliferative diseases.

Based upon another aspect of the present invention, what is provided are the antibodies for these polypeptides.

Based upon another aspect of the present invention, what is provided are antagonists which inhibit said polypeptides.

Based upon another aspect of the present invention, what is provided are mutations of the nucleic acid sequences of the present invention, along with diagnostic methods for diseases or susceptibility to diseases related to the abnormal expression of the polypeptides of the present invention.

Based upon another aspect of the present invention, what is provided are methods for using the polypeptides of the present invention or polynucleotides which encode said polypeptides in scientific research, DNA synthesis and artificially constructed DNA vectors.

The various aspects given above and other related aspects are readily apparent to technical people working in the field, based upon the instructions herein.

The present invention was realized via the following process: Extract mRNA, reverse transcribe into cDNA and construct a human aortic cDNA library. Obtain Fwa267 gene fragments from said library, and splice EST to obtain the full-length cDNA sequence of Fwa267. Study the expression and distribution of the Fwa267 gene in different tissues, along with the activity and functions of Fwa267 polypeptides.

### DETAILED DESCRIPTION OF THE INVENTION

Based upon one aspect of the present invention, what is provided is an isolated nucleic acid (polynucleotide) sequence such as the inferred amino acid sequence shown in SEQ ID NO:2. The polynucleotide of the present invention was discovered in the adult human aortic cDNA library. It is positioned on the No. 11 chromosome of the cell. It has an open reading frame so that it can encode polypeptide containing 370 amino acid residues. Homology analysis reveals that Fwa267 polypeptide is only 46% homologous to the secreting growth factor fallotein.

The inferred Fwa267 protein is formed from 370 amino acids. Its sequence characteristics are: (A) 276 to 279 Aa (NYSV) are N-glycosylation sites; (B) 268 to 271 Aa (KRYS) are cAMP and cGMP dependent protein kinase sites; (C) 262 to 270 Aa (RLNDDAKRY) are tyrosine kinase sites; (D) 1 to 61 Aa (MHRLIFVYTLICANFCSCRDTSATPQSASIKALRNANLRRDESNHLTDLYRRD ETIQVKGN) are TonB dependent receptor protein signals No. 1; (E) 100 to 105 Aa (GLEEAE), 192 to 197 Aa (GVSYNS), 303 to 308 Aa (GGNCGC), 304 to 309 Aa (GNCGCG) are myristic acylate sites; (F) 17 to 19 Aa (SCR), 29 to 31 Aa (SIK), 66 to 68 Aa (SPR), 80 to 82 Aa (TWR), 150 to 152 Aa (TFK), 243 to 245 Aa (TPR), 273 to 275 Aa (TPR), 320 to 322 Aa (SGK) are protein kinase C sites; (G) 17 to 20 Aa (SCRD), 168 to 171 Aa (SLLE), 181 to 184 Aa (TNWE), 199 to 202 Aa (SVTD), 219 to 222 Aa (TVED), 231 to 234 Aa (SWQE), 250 to 253 Aa (SYHD), 256 to 259 Aa (SKVD) are casein kinase II sites.

The polynucleotides of the present invention can be in the form of RNA or DNA. DNA includes cDNA, genome DNA and synthesized DNA. DNA can be double-stranded or single-stranded. If it is single-stranded, it can be coding strand or non-coding strand (antisense). The encoding sequence for mature polypeptide can be the same as that (107 to 1219 nucleotides) shown in SEQ ID NO: 1 (full-length 3739 nucleotides). Or, due to the abundance or degeneracy of the genetic code, the encoding sequence can also be different from that shown in SEQ ID NO: 1.

The encoding for the polynucleotide of the mature polypeptide shown in SEQ ID NO:2 can include: the encoding sequence for mature polypeptide; the encoding sequence and additional encoding sequences for mature polypeptide, such as encoded polypeptide leading sequence or secreted sequence polynucleotide; and an encoding sequence (along with a randomly added encoding sequence) and non-encoding sequence for mature polypeptide, such as introns or the non-encoding sequences of mature polypeptide encoding sequences 5′ and/or 3′.

Therefore, the term “polypeptide-encoding polynucleotide” includes polynucleotides containing only the polypeptide encoding sequence and polynucleotide containing additional encodings and/or non-encoding sequences.

The present invention also relates to variants of the polynucleotides referred to above. They encode inferred fragments, analogs and derivatives containing amino acid sequences shown in SEQ ID NO:2. Said variants can be naturally produced allelic variants of said polynucleotides or non-naturally produced variants. As is well-known in this field, allelic variants are a different form of polynucleotide, and can have one or many nucleotides substituted, deleted or added without substantially changing its encoding of polypeptides.

Therefore, the present invention includes polynucleotides able to encode the same amino acid sequences as contained in the mature polypeptides shown in SEQ ID NO:2, and also includes polynucleotide variants able to encode the fragments, derivatives and analogs of the mature polypeptide shown in SEQ ID NO:2. These include deficient variants, substitution variants and addition or insertion variants.

The present invention also includes said types of polynucleotides wherein the encoding sequences of mature polypeptides can be in the same reading frames, and can fuse with polynucleotides (such as those producing leading sequences) which assist polypeptide expression and secretion from the host cell. Leading sequences work as secretion sequences and control the transfer of polypeptides from the cells. Polypeptides with leading sequences are preproteins; when leading sequences are cut from host cells, mature polypeptides can be produced. The polynucleotides of the present invention can also encode proproteins, which are mature proteins containing prosequences; they are mature proteins with amino acid residues added to 5′, a type of inactive form. After removing the prosequences, active mature proteins can be produced.

Therefore, the polynucleotides of the present invention can encode a type of mature protein, and can also encode protein containing prosequences. They can also encode proteins containing both prosequences and presequences.

The polynucleotides of the present invention also include an encoding sequence blended with a marker sequence in the same reading frame. The marker sequence can be used to purify the polypeptides of the present invention. For example, when the host cell is bacteria, marker sequences can be provided by a pQE-9 vector and used to purify the 6-histidine of the fused product. Or, when the host is the cell of a mammal (such as a COS-7 cell) the marker sequence can be hemagglutinin (HA). The HA label should be an epitope derived from influenza hemagglutinin protein (Wilson et al., 1984, Cell 37:767).

The term “gene” refers to DNA fragments related to the production of polypeptides. It includes regions before and after the encoding segment, as well insertion sequences (introns) in the middle of the encoding segments (exons).

The full-length gene fragments of the present invention can be used as hybridization probes for the cDNA library. They separate full-length genes and genes highly homologous to or with similar biological activity to said full-length genes. Said probes preferably have at least 30 bases, though they can have up to 50 or even more. Said probes can also be used to identify cDNA clones corresponding to the full-length transcriptions and one or more genomic clones containing complete genes. The complete genes contain regulatory sequences, promoter sequences, exons and introns. For example, based upon the synthetic oligonucleotide probes of DNA sequences already known, the encoded portion of a gene can be isolated. Oligonucleotide probes, complementary to and tagged to the gene sequence of the present invention, can be used to screen hybridized library members from out of human cDNA, genome DNA or mRNA libraries.

The present invention also relates to hybridized nucleic acid sequences of the polynucleotides of the present invention. Two sequences have at least 85% homology, preferably at least 90%, and even more preferably at least 95%. The present invention especially relates to polynucleotides hybridized from the polynucleotides of the present invention under strict conditions. The term “strict conditions” used here means that hybridization occurs only with at least 95% homology between sequences, and preferably, at least 97%. The hybridized polynucleotides referred to above can be encoded with polypeptides with the same biological functions and activity as the mature polypeptides of the present invention.

As well, polynucleotides with the same homology as those of the present invention and can be hybridized can have at least 20 bases, preferably at least 30, and more preferably at least 50. They may or may not retain activity. These types of polynucleotides can be used as SEQ ID NO: 1 probes to recycle polynucleotides, or as probes used in diagnosis or as PCR primers.

Therefore, the present invention relates to polynucleotides and their fragments having at least 85%, preferably at least 90%, and more preferably at least 95% homology with the polynucleotides encoding the polypeptide shown in SEQ ID NO:2 (said fragments have at least 30 bases, preferably at least 50), as well the polypeptides encoded by said polynucleotides.

Based upon another aspect of the present invention, the present invention relates to inferred fragments, analogs and derivates of polypeptides with amino acid sequences shown in SEQ ID NO:2.

The terms “fragments,” “derivatives” and “analogs,” when related to SEQ ID NO: 1 encoded polypeptides or polypeptides with amino acid sequences such as those shown in SEQ ID NO:2, refer to polypeptides which basically retain the biological functions or activity of said polypeptides. Analogs can include proprotein, which when partially removed can produce active mature polypeptides.

The polypeptides of the present invention can be recombinant, natural or synthetic, though preferably recombinant.

Said polypeptides (SEQ ID NO:2) and their fragments, derivates or analogs can be: (i) a type of polypeptide with one or multiple amino acid residues substituted by conserved or non-conserved amino acid residues (preferably conserved amino acid residues), and the substituted amino acid residues may or may not be encoded by a genetic codon; or (ii) a type of polypeptide comprised of one or multiple amino acid residues containing a substituent; or (iii) a type of polypeptide comprised of a mixture of mature polypeptides with another type of compound, such as an increased half-life polypeptide compound (e.g. polyethylene glycol); or (iv) a type of polypeptide comprised of a mature polypeptide mixed with an added amino acid, such as a leading or secreting sequence, or such as a sequence used to purify mature polypeptide. Under the guidance of this text, these types of fragments, derivatives and analogs will be familiar to technical personnel in this field.

The polypeptides and polynucleotides provided in the present invention are preferably in isolated form, and preferably purified to an even consistency (homogenesis).

The term “isolated” refers to when said substances are separated from their original environment (e.g. if said substances occur naturally, then it refers to their natural environment). For example, polynucleotides or polypeptides naturally occurring in living animals are not isolated, but the same polynucleotides or polypeptides separated from a portion or total of a concurrent substance in a natural system are considered isolated. These kinds of polynucleotides can be part of a vector, and these kinds of polynucleotides or polypeptides can be part of a composition if this type of vector or composition is not part of its natural environment.

The polypeptides of the present invention include those shown in SEQ ID NO:2 (especially mature polypeptide), as well as those having at least 85% homology, preferably at least 90% homology and more preferably at least 95% homology with polypeptides shown in SEQ ID NO:2. The present invention also includes the fragments of said polypeptides, which typically contain at least 30, preferably at least 50, amino acids.

As is familiar to those in this field, homology between two polypeptides is determined by a comparison of the amino acid sequence of one polypeptide and its conserved amino acid substitute with those of another polypeptide.

When polypeptide is synthesized, the fragments (or partial polypeptide) of the present invention can be used to produce full-length polypeptide. Said fragments can be used as media in the production of full-length polypeptides. Similarly, the polynucleotide fragments of the present invention can be used to synthesize the full-length polynucleotides of the present invention.

Based upon another aspect of the present invention related to the vectors containing the nucleotides, the host cells engineered from the vector genes are used along with recombinant techniques to produce the polypeptides of the present invention.

The host cells are produced by genetic engineering (transduction, transformation or transfection) of the vectors of the present invention. Said vectors can be clones or expressed vectors. Vectors can be in the form of plasmids, virus particles and bacteriophages. Host cells can be engineered by promoters, screened transformers or in a conventional nutrient culture base for augmenting the Fwa267 gene of the present invention. The conditions for cultivation (temperature, pH value) are determined separately for each type of host cell, which is readily apparent to technical personnel in this field.

Recombinant techniques can be used on the polynucleotides of the present invention to produce polypeptides. Polynucleotides can be included in any type of vector suitable for expressing polypeptides. These kind of vectors include chromosomal, non-chromosomal and synthetic DNA sequences. For example, the SV40 derivative, bacteria plasmids, bacteriophages, baculoviruses, yeast particles, vectors resulting from the binding of particles and bacteriophage DNA, virus DNA (such as that in cowpox, adenovirus, fowlpox virus and pseudorabies). Other vectors can also be used as long as they can reproduce and survive in the host cell.

Many different methods can be used to insert suitable DNA sequences into vectors. Generally speaking, the conventional method known to those in the field is inserting DNA sequences into suitable restriction endonuclease sites.

A transport can be linked to a suitable expression control sequence (promoter) to express its DNA sequence, as a means of guiding the synthesis of mRNA. Examples of promoters are: LTR or SV 40, colon bacilli lac or trp, bacteriophages λ P_{L}, as well as other known promoters, control prokaryotic or eukaryotic cells or promoters expressed by genes in viruses. Expression vectors can also include leading ribosome binding sites which initiate translation, and transcription terminators. Vectors can also include suitable sequences for augmenting expression.

Preferable expression vectors contain one or multiple selectable marker genes so that post-transformation screening of the host cells provides a phenotype. Examples include using the eukaryotic host cell's dihydrofolate reductase or neomycin resistance, or using colon bacilli's tetracycline and ampicillin resistance.

Vectors containing the suitable DNA sequences above with suitable promoters or regulatory sequences can be used to transform suitable hosts so that they express protein.

Representative examples of suitable hosts are: bacteria cells, such as Escherichia coli, streptomyces and S. typhimurium; fungal cells, such as yeast; insect cells, such as Drosophila S2 and Spodoptera Sf9; animal cells, such as CHO, COS and Bowes melanoma; adenoviruses; and plant cells. As per the instructions herein, choosing appropriate hosts is well within the scope of knowledge of technical personnel in this field.

More practically speaking, the present invention also includes recombinant structures containing one or multiple sequences as described above. These structures include vectors inserted forward or backward into the nucleic acid sequences of the present invention, such as plasmids or virus vectors. In a more ideal implementation the structure would include operative regulatory sequences linked to said sequences, such as promoters. Many suitable vectors and promoters are familiar to people in this field, and they can be obtained commercially. Examples of bacterial vectors are: pQE70, pQE60, pQE-9 (Qiagen), pBS, pD10, phagescript, psiX174, pbluescript SK, pbsks, pNH8A, pNH16a, pNH18A, pNH46A (Stratagene), ptrc99a, pKK223-3, pKK233-3, pDR540 and pRITS (Pharmaca); examples of eukaryotic vectors are: pWLNEO, pSV2CAT, pOG44, pXT1, pSG (Stratagene), pSVK3, pBPV, pMSG and pSVL (Parmacia). Other plasmids or vectors may be used if they can reproduce and survive in the host.

Vectors with CAT (chloramphenicol transferase) or other selectable markers can be chosen from out of the promoter region. Two suitable vectors are PKK232-8 and PCM7. Especially notable bacterial promoters include lacI, lacZ, T3, T7, gpt, λP_{R}, P_{L} and trp. Eukaryotic promoters include CMV immediate early, SV thymidine kinase, early and late SV40, LTRs from retroviruses and mouse metallothionein-I. Selecting suitable vectors and promoters are well within the scope of knowledge of technical personnel in this field.

In another implementation, the present invention relates to a host cell containing the structures mentioned above. The host cell can be high-grade eukaryotic cells (such as mammal cells), low-grade eukaryotic cells (such as yeast cells) or prokaryotic cells (such as bacterial cells). Calcium phosphate transfection, DEAE-dextran mediated transfection or electroporotion can be used for integration of the structure into the host cell (Davis, L., Dibner, M., Mattey, I., 1986, Basic Methods in Molecular Biology).

Conventional methods can be used on the structure in the host cell to make a product from recombinant sequence encoding. In fact, the polypeptides of the present invention can be synthesized using a standard polypeptide synthesizer.

Under the control of a suitable promoter, mature protein can be expressed in mammal cells, yeast cells, bacterial cells or other cells. Originating from the DNA structure's RNA of the present invention, an acellular translation system can produce the desired protein as well. Sambrook et al., in Molecular Cloning: A Laboratory Manual (Vol. 2, A Cold Spring Harbor Laboratory Press, 1989), describe suitable clones and expressed vectors using prokaryotic and eukaryotic hosts.

Enhancer sequences inserted into vectors can improve transcription of the encoding of the polypeptides of the present invention by the high-grade eukaryotic cells. Enhancers are the cis-acting components of DNA, and are generally 10 to 300 bp. They are promoters which improve transcription. Examples of enhancers are: SV40 enhancers with a reproductive starting point upstream 100 to 270 bp, many forms of tumor enhancers and adenovirus enhancers.

Generally, recombinant expressed vectors include reproductive starting points and screened marker genes (e.g. Escherichia coli's ampicillin resistance genes and Saccharomyces cerevisiae's TRP1 gene), and are also obtained from highly-expressed genes, with the ability to guide promoters transcribed by structural genes downstream. These types of promoters can be obtained by encoding operons like glycolytic enzymes (such as 3-phosphoglycerate kinase (PGK)), α-factor, acid phosphatase or heat shock protein. Hetero sequences can be assembled in appropriate ways to translate initiation sequences and terminal sequences. Preferably, they are assembled with secreted leading sequences which are able to guide proteins to the cell's periplasm or outer nutrient medium. Hetero sequences can encode fusion protein containing N-terminus peptide recognition. Said peptide recognition has ideal characteristics, such as stable expression of recombinant products and a simplified purification process.

By inserting the structured gene of the target protein, the suitable translation initiation and termination signals and the functional promoter together, you can construct an expressed vector useful for bacteria. Said vector contains one or multiple selectable markers and a reproduction starting point to maintain it and augment it in the host when necessary. The prokaryotic hosts suitable for transformation include many types of Escherichia coli, Bacillus subtilis, S. typhimurium, pseudomonas, streptomyces and staphylococcus.

A representative, though not limited, example, is that the expressed vector of a bacteria can contain the selectable marker and reproductive starting point of vectors sold on the market. These commercially available vectors include genetic components of the well-known clone pBR322 (ATCC37017). Other commercially available vectors include pKK223-3 (Pharmacia Fine Chemicals, Uppsala, Sweden) and GEM1 (Promega Biotec, Madison, Wis., U.S.A.). The pBR322 “skeleton” is fused with suitable promoters and structural sequences awaiting expression.

For transformation into a suitable host bacterial strain, wait until it has grown to a suitable cell density and use an appropriate method (such as temperature variation or chemical induction) to induce the selected promoter. Continue to cultivate the cell for a period of time. The centrifugal method for harvesting cells is common, as are either physics or chemistry methods for breaking up cells. The crude product is retained for further purification. Any type of conventional method can be used to break up the microbial cells, such as freezing and thawing, ultrasonic treatment, mechanical disruption or splitting solutions. These methods are familiar to technical personnel in this field.

Various types of cultivation systems for mammal cells can be used for expression recombinant proteins. Examples include the monkey kidney fibroblast COS-7 cell line described by Gluzman (Cell 23:175 (1981)) and other cell lines able to express compatible vectors, such as the C127, 3T3, CHO, HeLa and BHK cell lines. Mammal expressed vectors contain reproductive starting points, suitable promoters and enhancers, as well as ribosome fusion sites, polyadenylation sites, splice donors and receptor sites, transcription termination sequences and 5′ flank non-transcription sequences. DNA sequences obtained from SV40 virus genomes, such as SV40 reproduction starting points, early promoters, enhancers splice and polyadenylation sites, can be used to provide the necessary non-transcription genetic components.

Many different methods can be used to recycle the polypeptides of the present invention from out of the recombinant cell cultivators and to purify them, including ammonium sulfate or ethanol precipitation, acid extractants, anion or cation exchange chromatography, phosphocellulose chromatography, hydrophobic interaction chromatography, affinity chromatography, hydroxylapatite chromatography, phytohemagglutinin chromatography and high performance liquid chromatography (HPLC).

The polypeptides of the present invention can be purified naturally, or chemically synthesized, or prepared using recombinant techniques from prokaryotic or eukaryotic hosts (such as bacteria, yeast, high-grade plants, cultivated insects or mammal cells). Based upon the hosts used in the recombinant methods, the polypeptides of the present invention can be either glycosylated or non-glycosylated. Said polypeptides can also contain one initiated methionine residue.

The polynucleotides and polypeptides of the present invention can be used as research reagents and materials in the treatment and diagnosis of human diseases. Fwa267 can inhibit proliferation of heart muscle and smooth muscle cells. In normal conditions they may participate in maintaining the terminal differentiation of myocytes. Overly-expressed Fwa267 may induce death in myocytes (e.g. ventricular aneurysm), while low levels of expression may lead to increased myocyte growth.

Based upon another aspect of the present invention, what is provided is a method for identifying activators or antagonists for the polypeptides of the present invention. One method is to use an existing compound: after detecting said compound's ability to enhance or block Fwa267 polypeptide's functioning with a receptor, it produces a second messenger. The second messenger includes, but is not limited to, the following: protein tyrosine kinase (PTK), cAMP guanylate cyclase, ion channel or inositol phosphate hydrolysis. Another method for identifying the polypeptide antagonist is competitive inhibition, which takes a compound when it does not exist and compares it with the Fwa267 polypeptide molecule count when fused with the receptor. For testing said compound when it exists, the variation with the Fwa267 polypeptide molecule count when fused with the receptor is used to determine potential antagonists.

The potential antagonist includes antibodies, or in a few cases includes oligopeptides fused with the polypeptides of the present invention, which fuses with said polypeptides and effectively eliminates its function.

Another potential antagonist compound uses antisense structures prepared using antisense techniques. Using a triple helix to form antisense DNA or RNA to control expression of the gene, said methods are based upon the fusion of polynucleotides with either DNA or RNA. For example, nucleic acid sequence 5′ of the mature polypeptides of the present invention can be encoded to design antisense RNA approximately 10 to 40 base pairs in length. Or, one can design DNA complementary to a gene region related to transcription (Triple helix: see Lee et al., 1979 Research in Nucleic Acid 6:3073; Cooney et al., 1988 Science 241:456; and Dervan et al., 1991 Science 251:1360), to block transcription as well as production of the polypeptides of the present invention. Antisense RNA hybridizes with mRNA within the body, and blocks mRNA molecule translation to become the polypeptide of the present invention (Antisense: Okano, J., 1991 Journal of Neurochemistry 56:560; Oligodeoxynucleotide as a Gene Expression Antisense Inhibitor (CRC Publishing, Boca Raton, Fla., U.S.A. 1988)). Said oligonucleotides can be transmitted to the cells, and can express antisense RNA and DNA within the body as a means of inhibiting the production of the polypeptides of the present invention.

Antagonists also include small molecules, which are fused with the polypeptides of the present invention to prevent said polypeptides from working with their receptors. This blocks their normal biological activity. Small molecules include, but are not limited to, small peptides or peptoid molecules.

The activators and antagonists of the polypeptides of the present invention can fuse with suitable vectors to form pharmaceutical compositions. These compositions contain amounts of polypeptide and pharmaceutically acceptable vectors or excipients effective for medical treatment. Vectors include, but are not limited to, saline, buffer, glucose, water, glycerol, ethanol and composites of said substances. Formulation should be compatible with pharmaceutical prescriptions.

The present invention also provides a type of drug packaging or reagent kit, including one or multiple containers in which one or multiple groups of the pharmaceutical composition of the present invention are given. What can also be provided is information regarding approval by governmental drug administration authorities, along with that regarding the relevant manufacture, use and sale of the drugs or biological products. The pharmaceutical composition of the present invention can also be used in conjunction with other compounds used for treatment.

This pharmaceutical composition can be administered in the normal ways, such as orally, topically, intravenously, intraperitoneally, intramuscularly, subcutaneously, intranasally and intracutaneously. Appropriate amounts are given for treatment and/or prevention of specified diseases. Generally, at least 10 gammas per kilogram of body weight is given. In most cases daily dosage does not exceed 8 milligrams per kilogram of body weight. In most cases, and in consideration of the method of administration and symptoms, daily dosage is from about 10 gammas per kilogram of body weight to 1 milligram per kilogram of body weight.

Based upon another aspect of the present invention, the polypeptides of the present invention and their activators and antagonists can be expressed within the body, a method commonly known as “gene therapy.”

Therefore, genetic engineering of the patient's cells can be undertaken by encoding the polypeptide nucleic acid of the present invention (DNA or RNA) outside the body. Engineered cells can then be provided to the patient. Said method is familiar to those within the field. For example, the retrovirus containing the encoded RNA of the polypeptides of the present invention can be used for genetic engineering of cells.

Similarly, well-known methods can be used for genetic engineering of cells within the body in order to express polypeptide within the body. The retrovirus containing the encoded RNA of the polypeptides of the present invention can be used for encasing cell transduction to enable the production of the infectious virus particles of the targeted gene. Use in patients can engineer cells within the body and express said polypeptides. Under the guidance of the present invention, said methods are well-known to technical personnel working in this field.

Retroviruses obtained from retrovirus particle vectors include, but are not limited to: the Moloney murine leukemia virus, the spleen necrosis virus, the retrovirus Rous sarcoma virus, the Harvey sarcoma virus, the avian leukosis virus, the gibbon ape leukemia virus, the human immunodeficiency virus, the adenovirus, myeloproliferative sarcoma virus and the breast tumor virus.

Said vectors contain one or multiple promoters. Suitable promoters include, but are not limited to: the retrovirus LTR; the SV40 promoter; the human cytomegalovirus (CMV) promoter (as described by Miller et al., 1989 Biotechnology Vol. 7, No. 9 980-990); or other promoters (such as eukaryotic cell promoters, including, but not limited to, histones, pol III and β-actin promoters). Other virus promoters which can be used include, but are not limited to, adenovirus promoters, thymidine kinase (TK) promoters and B19 parvovirus promoters. With the instructions herein, suitable promoters on selected vectors are well-known to technical personnel working in this field.

Suitable promoters are needed to control encoding of the nucleic acid sequences of the polypeptide of the present invention. They include, but are not limited to: adenovirus promoters (such as the main late promoters of the adenovirus); or heterologous promoters (such as cytomegalovirus (CMV) promoters); respiratory syncytial virus (RSV) promoters; inducible promoters (such as MMT promoters and metallothionein promoters); heat shock promoters; albumin promoters; ApoAI promoters; human globin promoters; viral thyrnidine kinase promoters (such as Herpes simplex thymidine kinase promoters); retrovirus LTRs (including the modified retrovirus LTRs described above); β-actin promoters; and human growth hormone promoters. Promoters can also be natural promoters encoding the polypeptide genes referred to above.

Retrovirus particle vectors can be used to transduce encasing cells in order to produce producer cells. Transfectable encasing cells include, but are not limited to: PE501, PA317, ψ-2, ψ-AM, PA12, T19-14X, VT-19-17-H2, ψCRE, ψCRIP, GP+E-86, GP+envAml2 and DNA cell lines (as described by Miller in Human Gene Therapy Vol. 1 pp. 5-14 (1990)—the contents of that study are fully referenced in this patent). Vectors can use any method already known in this field to transduce encasing cells. These methods include, but are not limited to: electroporotion, liposome and CaPO₄ precipitation. Retrovirus particle vectors can be embedded in liposome, or can be coupled onto liposome, and then guided into the host.

Producer cells produce infectious retrovirus vector particles. Said particles contain nucleic acid sequences which can encode said polypeptides. These retrovirus vectors can be used within or without the body to transduce eukaryotic cells. The transduced eukaryotic cells will express the nucleic acid sequences which encode said polypeptides. Transducible eukaryotic cells include, but are not limited to: embryonic stem cells, embryonic cancer cells, hemopoiesis stem cells, hepatocytes, fibroblasts, myoblasts, keratinocyte, endothelial cells and bronchial epithelial cells.

Based upon another aspect of the present invention, the present invention relates to the use of the Fwa267 gene in diagnosis and testing. Testing of mutations in the Fwa267 nucleic acid sequence can diagnose related diseases or susceptibility to diseases.

Many techniques can be used at the level of DNA to test for mutating entities carrying the Fwa267 gene. They can come from the patient's cells, such as those from the blood, urine, saliva, check of living tissues or dissected corpses. Genome DNA can be used directly in tests, or PCR amplification can be used before analysis (Saiki et al., 1986 Nature 324:163-166). RNA or cDNA can also be used for this purpose. For example, PCR primer, which is complementary to the polynucleotides of the present invention, can identify and analyze mutations. When compared with normal genotypes, examination of deletions and insertions is based upon the changed size of the amplified product. Hybridization of amplified nucleic acid sequences with radio-labeled RNA or antisense DNA can be used to identify point mutation. With RNaseA digestion or via the difference in melting temperature, one can discriminate between completely paired sequences and mismatched double strands.

DNA sequencing can directly reveal differences in sequence between genes and mutation-carrying genes. Also, cloned DNA fragments can act as probes to examine specific DNA segments. When fused with PCR, this method's sensitivity is improved greatly. For example, sequencer primers and double-stranded PCR products or single-stranded template molecules produced by improved PCR methods can be used together. Conventional automatic sequencing methods use radio-labeling or fluorescent-labeling to confirm nucleic acid sequences.

Genetic testing of differentiation in DNA sequences can mean testing of gel containing or not containing a denaturant to reveal changes in the DNA fragments' electrophoretic mobility. Small sequence deletions or insertions can be revealed from electrophoresis of the high distinguishability gel. DNA fragments with different sequences can be differentiated on denaturing formamide gradient gel. Based upon its specified melting point or partial melting temperature, different DNA fragments will stop at different places on the gel (see Myers et al. 1985 Science 230:1242).

RNase and S1-protected nuclease protection analysis or chemical cleavage can also test for sequence variations at specific places (Cotton et al. 1985 PNAS U.S.A. 85:4397-4401).

Therefore, one can use hybridization, ribonuclease protection, chemical cleavage, direct DNA sequencing or use restriction enzymes (such as restriction fragment length polymorphisms (RFLP)) with Southern blotting of genome DNA to test variations in DNA sequences.

Aside from more conventional gel electrophoresis and DNA sequencing, mutations can also use in situ analysis.

Based upon another aspect of the present invention, the present invention relates to a type of diagnostic analysis which tests changes in the content of Fwa267 polypeptides in different tissues. Based upon comparisons with normal tissues, too much expression of said polypeptides in certain tissues can reveal the existence of disease or susceptibility to disease. Analytical methods of testing the present invention's polypeptide content in samples extracted from hosts is familiar to technical personnel working in this field. They include radioimmunoassay, competitive binding assay, Western blotting analysis, enzyme-linked immunosorbent assay and “sandwich” assay, and the preferable ELISA test. For ELISA, the specific antibodies (preferably single clone antibodies) of the polypeptides of the present invention are first prepared. Then, the reporter antibodies of said single clone antibodies are prepared. The reporter antibodies are bound to a testable reagent, such as a radioactive reagent, fluorescent reagent or horseradish peroxidase. Samples are extracted from the host, and are incubated in a solid support (such as polystyrene blood) bound to a protein in the sample. Cultivated together with non-specific protein (such as bovine serum albumin), they cover any free protein binding sites in the blood. Following that, during the period in which the single clone antibodies are bound to any polypeptides of the present invention which are bound to the polystyrene blood, the single clones will cultivate in the blood. A buffer is used to wash out all of the single clone antibodies which do not bind. At this time, receptor antibodies connected to the horseradish peroxidase are placed in the blood, leading the receptor antibodies to bind to any of the single clone antibodies bound to the polypeptides of the present invention. Single clone antibodies not binding to anything are washed away. After this, peroxidase substrate is compared to a calibration curve, and within the time allotted the amount of color produced is the amount of protein existing in the allotted area in the patient sample.

Competitive assay can be used to test polypeptide content. The method includes binding the specific antibodies of Fwa276 polypeptides to a solid support, and then labeling (e.g. radio-labeling) the polypeptides of the present invention. The sample extracted from the host is put through the solid support, and then the labeling content is tested to determine the amount of competitive binding antibodies in the sample. This allows one to determine the polypeptide content in the sample.

The identification of chromosomes by the sequences of the present invention is extremely valuable. Said sequences specifically target certain positions of the human chromosome, and hybridize with them. Currently, there are only very few types of chromosome labeling reagents based on practical sequence databases (repeatable polymorphic) which can be used to label positions of chromosomes. Mapping based upon the DNA chromosomes of the present invention is the first step in linking these sequences with related diseases genes.

Put simply, with cDNA preparing PCR primers (preferably 15-25 bp) one can position the chromosome for the sequence. With computer analysis of the gene's 3′ translated region, a primer can be chosen very quickly. Primers should not go past the genome DNA's first exon or they will complicate amplification. Then, primer is used with PCR to screen somatic cell heterozygotes containing individual human chromosomes. Only heterozygotes containing genes related to said primers will produce amplified fragments.

PCR mapping of somatic cell heterozygotes is a quick method for positioning specific DNA on specific chromosomes. Based upon the present invention, using the same oligonucleotide primers and a group of fragments from specific chromosomes or genomic clones, using similar methods sub-positioning can be completed. Other methods of chromosome mapping include in situ hybridization, labeling, flow-sorted chromosomes used for pre-screening and using hybridization to do pre-screening; these are means of constructing chromosome-specific cDNA libraries.

Fluorescent in situ hybridization (FISH) of cDNA clones and metaphase chromosome smears can reveal the positions of chromosomes even better. This technique can use 50 or 60 base cDNA. See Verma et al.'s overview Human Chromosomes: Manual of Basic Techniques Pergamon Press, New York (1988).

Once the gene is positioned on the chromosome its physical position on the chromosome can be linked to the data on the genetic map. This data can be found in V. McKusick's Mendelian Inheritance in Man (accessed via the internet at the Welch Medical Library site of Johns Hopkins University). Then, genetic linkage analysis (collective heritage of physically neighboring genes) is used to determine the relationship between the gene and the disease which has already become fixed in the same region as the chromosome.

After that the difference between the cDNA or the genome sequence and the diseased entity and normal entity needs to be determined. If mutation is observed in a portion or the whole of the diseased entity, and not in the normal entity, said mutation may be the factor in the disease.

Said polypeptides, their fragments, derivatives or analogs, or cells expressing the above-mentioned substance can be used as an immunogen to produce antibodies. Antibodies can be polyclonal or monoclonal. The present invention also includes chimeric, single-stranded and humanized antibodies, as well as Fab fragments or the products of Fab-expressed libraries. Many well-known methods in this field can be used to produce these antibodies and fragments.

Directly infusing animal bodies (preferably non-human bodies) or using the polypeptides of the present invention, one can obtain relevant antibodies. These antibodies will fuse with said polypeptides. In this way, even sequences encoding polypeptide fragments can produce antibodies able to fuse completely natural polypeptides. These types of antibodies can isolate polypeptides from out of the tissue expressing said polypeptides.

Any technique for producing antibodies with consecutive cell line cultivation can be used to prepare monoclonal antibodies. Examples are the hybridoma technique (Kohler and Milstein, 1975 Nature 256:495-497), the trisome hybridoma technique, the human B-cell hybridoma technique (Kozbor et al. 1983 Immunology Today 4:72) and the EBV-hybridoma technique (Cole et al. 1985 “Monoclonal Antibodies and Cancer Therapy” Alan R. Liss, Inc., pp. 77-96).

The production technique for single-stranded antibodies (U.S. Pat. No. 4,946,778) could be improved to produce single-stranded antibodies to fight against the polypeptides of the present invention (which have immunogenicity). What could also be used is transgenic mice to express humanized antibodies to fight against said polypeptides.

In order to understand the substance of the present invention more clearly, please refer to the drawings and embodiments below. The drawings and embodiments are for explanation only, and do not limit the present invention in any way.

Under the guidance above, many improvements and variations are possible for the present invention. Therefore, within the scope of the claims, the present invention may be undertaken using methods different from those described above.

### BRIEF DESCRIPTION OF THE DRAWINGS

FIG. 1: Fwa267 distributed in normal tissue.

FIG. 1A shows result of hybridization of β-actin with MTN membrane.

FIG. 1B shows result of hybridization of Fwa267 with MTN membrane.

FIG. 2: Fwa267 distributed in various tumor cell lines.

FIG. 2A shows result of hybridization of β-actin with tumor cell line membrane. IG. 2B shows result of hybridization of β-actin with tumor cell line membrane.

FIG. 3: Effect of concentration of homocysteine upon smooth muscle cell expression Fwa267.

FIG. 3A shows the sample content.

FIG. 3B shows homocysteine stimulating Fwa267's expression at various times.

FIG. 4: Expression of Fwa267 in adult and fetus hearts.

FIG. 4A shows the sample content.

FIG. 4B shows Fwa267's expression.

FIG. 5: Expression of Fwa267 in adult, fetus and hypertrophic Tetralogy of Fallot myocytes.

FIG. 5A shows the sample content.

FIG. 5B shows Fwa267's expression.

FIG. 6: Expression of Fwa267 in a normal aorta and one with subacute heart failure, as well as in an animal heart with chronic heart failure.

FIG. 6A shows a normal animal aorta.

FIG. 6B shows an animal aorta with subacute heart failure.

FIG. 6C shows animal heart tissue with chronic heart failure.

FIG. 7: Expression of Fwa267 in normal heart tissue and myocardial infarction ventricular aneurysm tissue (negative control).

FIG. 8: Result of hybridization of Fwa276 protein electrophoresis and Western blotting.

FIG. 8A shows SDS-PAGE electrophoresis of Fwa267.

FIG. 8B shows hybridization of Fwa267 protein with Western blotting.

### EMBODIMENTS

### Embodiment 1

### Construction of an Adult Aorta cDNA Library

1.1 Obtaining RNA

The RNA Gents® Total RNA Isolation System kit is available from Promega (Cat No. Z5110) of the U.S.A. The process is as follows: Measure out 0.3 g of adult human aorta tissue stored in liquid nitrogen, add a homogenate of 10 ml of denaturation solution (4 M guanidinium isothiocyanate, 25 mM trisodium citrate) and 1 ml of 2M sodium acetate (pH 4.0). Add an equal volume of water saturated phenol and 0.2 times volume of chloroform, shake well for 15 seconds and then place on ice for 15 minutes. Put through 10,000 rpm centrifugal force for 20 minutes at 4° C. Add an equal volume of isopropanol and store for 2 hours at −20° C. Centrifuge. Use 5 ml of denaturation solution to suspend and precipitate, and then repeat the steps above. Precipitate using 1 ml pre-cooled 75% alcohol wash, and evaporate trace amounts of alcohol for 5-10 minutes. Dissolve RNA with deionized water treated with diethyl pyrocarbonate (DEPC).

1.2 Isolation of mRNA

Mature mRNA 3′ contains a Poly(A) formed from 20-250 adenylates. Based upon this characteristic, affinity chromatography analysis can be used to isolate mRNA and its RNA. The oligo(dT) cellulose in this test contains 12-18 nucleotide polymer strands. Under conditions of high salinity, the oligo(A) tail mRNA and oligo(dT) fuse and remain on the column, while oligo(A) tail rRNA and tRNA are washed away. A low saline solution is used to wash away the mRNA still on the column.

The Quick Prep® Micro mRNA Purification kit as available from Pharmacia of Sweden. In 1.5 ml centrifuge tube #1, not containing the RNA enzyme, add 1 ml oligo(dT) cellulose, suspended. In 1.5 ml centrifuge tube #2 add 1 ml of sample buffer-diluted RNA. Tube #1 and tube #2 are centrifuged for 1 minute at 12,000 rpm at room temperature. Suck out supernatant from tube #1, and put supernatant from tube #2 into tube #1, and lightly agitate for 5-10 minutes. Centrifuge at room temperature for 10 seconds at 12,000 rpm. Wash 5 times with 1 ml high saline buffer (10 mM Tris-HCl pH 7.5, 1 mM EDTA, 0.5 M NaCl), then centrifuge. Wash 5 times with 1 ml low saline buffer (10 mM Tris-HCl pH 7.5, 1 mM EDTA, 0.1 M NaCl), then centrifuge. Precipitate with 0.3 ml low saline buffer with suspended oligo(dT) and spin until a small amount is left on the centrifuge column. Centrifuge at 12,000 rpm for 5 seconds. Wash three times with 1.5 ml low saline solution, then centrifuge. Use 2×0.2 ml eluent to collect the mRNA. Use a spectrophotometer to determine optical density, calculated as OD260/280. Obtain 300 μl of mRNA, add 7.5 μl glycogen, 30 μl 2.5M potassium acetate (pH 5.0), 750 μl ethyl alcohol absolute and store at −70° C. Centrifuge for 5 minutes before use, 75% ethyl alcohol wash, DEPC-treated water soluble mRNA.

1.3 Synthesis of cDNA

For synthesis process refer to the instructions in ZAP Express™ cDNA Synthesis Kit*and ZAP Express™ cDNA Gigapack® II Gold Cloning Kit* (Stregene of U.S.A., Catalog No. 200403 and 200404).

In a 0.5 ml centrifuge tube add in succession 5 μl 10×first strand buffer, 5 μl methylated dNTP mixture, 2 μl Xho connexon oligo(dT)18 primer (1.4 μg mRNA and let sit for 10 minutes at room temperature. Then, add 1.5 μl Moloney murine leukemia virus (MMLV) reverse transcriptase (50 u/μl) for a total reaction volume of 50 μl. Extract 5 μl and transfer to another centrifuge tube, and add 0.5 μl α-³²P deoxyadenosine triphosphate (dATP) (800 ci/mmol) to determine the mass of the first strand synthesis. The reaction is performed at 37° C.

The DNA/RNA hybrid molecules work in the DNA polymerase I to synthesize the second strand, with the first strand as a template. Into an ice bath add, in succession, 45 μl first strand cDNA, 20 μl 10×second strand buffer, 6 μl dNTP mixture, 114 μl deionized water, 2 μl ribonuclease H (1.5 u/μl), 1 μl DNA polymerase I (9.0 u/μl), for a total volume of 200 μl. Mix evenly and incubate at 16° C. for 2.5 hours. When the reaction is completed use phenol:chloroform (1:1) to extract the alcohol precipitate.

1.4 Linking cDNA with Vectors

Take out of the reagent kit 9 μl EcoRI connexon (sequence: 5′□AATTCGGCACGAG□3′ and 3□GCCGTGCTC□5′) and dissolve the precipitate. Extract 1 μl for electrophoresis to determine the synthetic mass of the first and second strands. Into the remaining 8 μl second strand cDNA add in succession 1 μl 10×ligase buffer, 1 μl 10 mmol/Lγ-adenosine triphosphate (γ-ATP), 1 μl T4 DNA ligase (4 u/μl), and let it bathe in water at 8° C. for 16 hours. Then cultivate it at 70° C. for 30 minutes. Use restriction enzyme XhoI to digest for 1.5 hours. Sepharose C1-2B is put through the column to isolate, and remove less than 400 bases of nucleotide. This increases the proportion of full-length cDNA within the cDNA library.

1.5 cDNA Clone into ZAP Bacteriophage Vector

Polyclonal sites on the ZAP bacteriophage vector (Stratagene, U.S.A.) allow insertion of nucleic acid fragments 10 Kb in length. After entering the host the plasmid portion can be cut off from the vector to form the plasmid vector Bluescript. Both sides of the polyclonal sites of said vector have T₇ and T₃ bacteriophage promoters, which can be used in sequence analysis and probe synthesis. Inserting cDNA fragment vectors can express fusion protein which has antigenicity and biological activity. A detailed description of the experiment is as follows:

In a centrifuge tube add 100 ng cDNA, 0.5 μl 10×ligase buffer, 0.5 μl 10 mmol/Lγ-ATP (pH 7.5), 1 ul ZAP bacteriophage vector (1 ug/ul), 0.5 ul T4 DNA ligase (4 u/ul), and add to deionized water to a total volume of 5 μl. Link at 12° C. for 16 hours.

The resulting product needs to be encased by protein in order to produce a recombinant bacteriophage with transfection activity. Quickly extract the encased protein from −70° C., dissolve, and within 25 ul of encased protein add 1 ul of the linked reactant. Mix evenly, and react at 22° C. for 2 hours. Add 500 ul SM buffer (0.1 M NaCl, 0.08 M MgSO₄.7H₂O, 0.05 M Tris-HCl, 0.01% gelatin) and 20 ul chloroform. This mixture is the primary cDNA library.

1.6 Calculating Potency of Positive Clones

Extract 5 ul from the primary cDNA library, and dilute with 45 ul SM buffer. Add 10 ul of the resulting solution to 200 ul competence XL1-Blue MRF host bacteria (OD 600=0.5). Dissolve at 37° C. for 20-30 minutes. Add 3 ml of upper level agarose, mix evenly, and spread NZY (0.09 M NaCl, 0.08 M MgSO₄.7H₂O, 0.5% yeast extract, 1% NZ amine A) on a flat base. Place it upside down, cultivate overnight at 37° C., and then count the clones on the base.

The potency of the cDNA library is expressed by pfu/ml. pfu/ml=(plaque-forming units×dilution multiple)×1000/dilution volume (ul). A cDNA library containing more than 1×10⁶ clones guarantees that it contains every single copy mRNA. The adult aorta cDNA library of the present invention contains 2.4×10⁶ independent recombinant clones.

1.7 Amplifying and Storing the cDNA Library

The cDNA library can be used directly in screening, but it is not stable. Non-natural bacteriophages are easily deactivated, so they need to be amplified so that they can be screened multiple times. Extract 5×10⁴ bacteriophage transfected XL1-Blue MRF host bacteria, spread over a base, and cultivate at 37° C. for 8-10 hours. Then, in a 150 mm dish add 8 ml SM buffer and cultivate overnight at 4° C. Centrifuge, collect the supernatant, and obtain the amplified cDNA library. Add 0.3% chloroform and store at 4° C. Storage for long periods of time requires adding 7% dimethyl sulfoxide (DMSO) and freezing at −70° C.

### Embodiment 2

### New Full-length cDNA Clones

2.1 Random Clones Polymerase Chain Reaction (PCR) Amplification

The cDNA library is spread on a plate, and plaque density is 200-500 clones per petri dish (150 mm). Pick a clear single bacteria and transfer into a bacteria-free centrifuge tube containing 75 ul SM buffer and 5 ul chloroform, and set at 4° C. Centrifuge evenly, and extract the supernatant liquid 5 ul, using ZAP bacteriophage vector 3′ primer (5′ CCAAGCTCGAAATTAACCCTCAC 3′) and 5′ primer (5′ CAGTCAATTGTAATACGACTCACT 3′) to undergo PCR amplification. The total volume of the reaction is 50 ul. The amplification parameters are 94° C. for 3 minutes before denaturing, then 94° C. for 45 seconds, 55° C. for 30 seconds, and 72° C. for 3 minutes for 30 cycles in total. Extend 72° C. for 5-10 minutes. Calculate PCR product content based upon brightness of electrophoresis in 1% agarose gel.

2.2 Sequence Assay of Expression Sequence Tag (EST)

ABI377 automatic sequencer (ABI PRISM™ 377DNA sequencer) and automatic sequencer reagent (BigDye™ Terminator Ready Reaction Mix) are available from PE in the U.S.A. Use dideoxy chain termination for sequencing following recommended conditions for use.

Non-radioactive CY5-fluorescein is used as a marker. Sequencer primer T₃ (5′ ATT AAC CCT CAC TAA AGG GA 3′) and T₇ (5′ TAA TAC GAC TCA CTA TAG GG3′) is used for sequencing. Each sample uses 5 pmol/ul of primer. The sequencing template is the PCR product, 30-100 ng. PCR amplification parameters are: 94° C. for 3-5 minutes, then 94° C. for 30 seconds, 50° C. for 15 seconds, 72° C. for 1 minute, for 20 cycles. Then, 94° C. for 30 seconds, 72° C. for 1 minute, for 15 cycles. Extend at 72° C. for 5 minutes. The DNA product is put through electrophoresis on a base with 8 mol/L urea and 6% polyacrylamide gel. Voltage is 1,500 V, power 24 W, electrophoresis at 250-300 minutes (ALF Express™ DNA sequence, Pharmacia of Sweden).

2.3 Bioinformatics Analysis of EST

Using the BLAST software program (Basic Local Alignment Search Tool) at the Bioinformatics Center in the U.S.A., each cDNA clone's EST is put through homologous comparison with the GenBank/EMBL/DDBJ database (http://www.ncbi.nlm.nih.gov). Based upon the BLAST standard (the standard for the majority of laboratories domestically and abroad), a new EST is one with homologous sequences smaller than 100-200 bases and score values of less than 100 sequences. The present invention's Fwa267 is new EST.

2.4 EST Primer Walking Sequencing

2.4.1 ZAP Bacteriophage Inner Circularization

The ZAP bacteriophage vector is able, within the host, to transfer the target fragment from the λ bacteriophage vector directly to the plasmid, to circularize into PBK-CMV (early promoter carrying the CMV virus) phagemid. Refer to instructions for circularization process (ZAP Express cDNA Synthesis kit and ZAP Express cDNA Gigapack™ Gold Doning kit).

Use 3 ul bacteriophage stored at 4° C. carrying new EST and 1 ul auxiliary bacteriophage (a type of mutant bacteriophage with an extremely low ability to copy itself, it can provide protease and capsid protein for copying and encasing of plasmid DNA in the host cell) to transfect 300 ul Escherichia coli XL1-Blue MRF strain (OD600=1.0). After obtaining single-stranded DNA, the Escherichia coli XLOLR strain (provided in the kit) is transfected. In the test tube add 5 ml of culture solution, 25 ul kanamycin, and cultivate at 37° C. for 12-16 hours.

2.4.2 Extracting and Assaying Plasmid

Use German company Qiagen's “QIAPrep Spin Miniprep” kit to extract plasmid. Centrifuge at 2,400 rpm at 4° C. for 10 minutes, and collect bacteria which has been cultivated overnight. Discard the supernatant, and add 25 ul buffer P1 (100 ul/ml RNaseA, 50 mM Tris/HCl, 10 mM EDTA, pH 8.0) suspended bacteria, and transfer into the sterilized 1.5 ml centrifuge tube. Add 250 ul buffer P2 (200 mM NaOH, 1% SDS) and overturn the supernatant a few times to fully mix evenly. Add 350 ul buffer P3 (3.0 M Kac, pH 5.5) and immediately shake the tube 4-6 times. Centrifuge at 12,000 rpm for 10 minutes (Sorvall Mc12 V centrifuge). Collect the supernatant and transfer it to a micro purifier column equipped with a collection tube (QIA prep Spin Miniprep). Centrifuge at 12,000 rpm for 30-60 seconds. Discard the collection tube, and QIA prep purifier column with a sterilized centrifuge tube at the bottom add 50 ul buffer EB (10 mM Tris-HCl pH 8.5) or deionized water. Keep in column bed for 1 minute longer, then centrifuge for 1 minute. Collect the purified DNA.

Into the centrifuge tube add 3 ul enzyme digestion buffer (10×), 1 ul NotI endoenzyme (15 u/ul), 1 ul ECoRI endoenzyme (15 u/ul), 1 ul BSA, 2 ul DNA, 22 ul deionized water, total volume is 30 ul. Incubate at 37° C. for 4-6 hours. Determine the size of the plasmid after digestion.

2.4.3 Assay of New Full-Length cDNA

Use U.S.A. PE ABI377 automatic sequencer and sequencer reagent kit BigDye™ Terminator Ready Reaction Mix to assay the sequence of the PBK-CMV positive clone.

The reactant contains BD 4 ul, BOB 2 ul (400 mM Tris-HCl, pH 9.0, 10 mM Mg Cl₂), primer 2 ul (5 pmol/ul), DNA template 30-100 ng. Add H₂O to bring the total volume up to 20 ul. Use walking method (step-by-step priming method) to infer the full-length sequence of the cDNA; the next round of sequencing primer is determined by the base at the end of the product obtained by sequencing in the previous round. Use oligo 14.0 software to design the PCR primer.

### Results

As shown in SEQ ID NO:1, Fwa276 cDNA full-length has 3,739 base pairs. Based upon the Kozak law, assaying their initiation codons are 107 to 109 nucleotides (ATG). Encoded sequences are 107 to 1,219 nucleotides, the termination codons are 1,217 to 1,219 nucleotides. BLAST analysis reveals that said genes are positioned at number 11 chromosome.

Protein homologous analysis reveals that homology between the Fwa267 protein and the secreting growth factor fallotein is 46%. The inferred Fwa267 protein is formed by 170 amino acids. Its sequence characteristics are: (A) 276 to 279 Aa (NYSV) are N-glycosylation sites; (B) 268 to 271 Aa (KRYS) are cAMP and cGMP dependent protein kinase sites; (C) 262 to 270 Aa (RLNDDAKRY) are tyrosine kinase sites; (D) 1 to 61 Aa (MHRLIFVYTLICANFCSCRDTSATPQSASIKALRNANLRRDESNHLTDLYRRD ETIQVKGN) are TonB dependent receptor protein signals No. 1; (E) 100 to 105 Aa (GLEEAE), 192 to 197 Aa (GVSYNS), 303 to 308 Aa (GGNCGC), 304 to 309 Aa (GNCGCG) are myristic acylate sites; (F) 17 to 19 Aa (SCR), 29 to 31 Aa (SIK), 66 to 68 Aa (SPR), 80 to 82 Aa (TWR), 150 to 152 Aa (TFK), 243 to 245 Aa (TPR), 273 to 275 Aa (TPR), 320 to 322 Aa (SGK) are protein kinase C sites; (G) 17 to 20 Aa (SCRD), 168 to 171 Aa (SLLE), 181 to 184 Aa (TNWE), 199 to 202 Aa (SVTD), 219 to 222 Aa (TVED), 231 to 234 Aa (SWQE), 250 to 253 Aa (SYHD), 256 to 259 Aa (SKVD) are casein kinase II sites.

### Embodiment 3

### Distribution of Fwa267 in Normal Tissue

3.1.1 Test Material

A multiple tissue membrane (MTN membrane) containing 12 types of tissue is available from Clontech in the U.S.A. It is used to examine the distribution of the Fwa267 gene in normal tissue. β-actin is a type of housekeeping gene expressed homogenously in many types of tissue. In this test it is used for comparison.

3.2 Northern Hybridization

Refer to Experimental Techniques in Modern Molecular Biology (LU, Shengdong, ed., Xiehe Medical University Press of China, Vol. 2, 1999) pp. 147-149, 202-205, 207-213. Detailed explanation is as follows:

3.2.1 Extracting Total RNA

Measure 0.5 g of tissue in a 50 ml centrifuge tube, add 10 ml GTC and mix until homogenous. Add an equal volume of water-saturated phenol and 0.2 volume [sic] chloroform, shake vigorously for 15 seconds and let sit on ice for 20 minutes. Centrifuge at 12,000 rpm at 4° C. for 25 minutes. Discard the supernatant. Use 5 ml GTC to dissolve again, and repeat the other steps. Wash with 1 ml pre-cooled 75% alcohol, dry, use DEPC-treated water to dissolve. Test OD260 (optical density) and OD 280.

3.2.2 Electrophoresis with Formaldehyde Denaturing Gel

Measure out 0.6 g agarose gel and add to 52.2 ml DEPC-treated water, heat to dissolve. After gel has cooled to 65° C. add 6.0 ml 10×MOPS, 1.8 ml formaldehyde. Extract 4.5 ul (40-60 ug) total RNA, add 2.0 ul 10×MOPS, 3.5 ul formaldehyde, 10 ul formamide and mix evenly. Denature at 65° C. for 15 minutes, then cool on ice for 2 minutes. Add 2.0 ul sample buffer and mix evenly. Perform pre-electrophoresis for 5 minutes at 50 V. After the dye has gone completely into the gel, lower voltage to 40 V and mix buffer every 10 minutes.

3.2.3 Transfer Membrane

Stop electrophoresis when bromphenol blue has moved to base of gel. Use DEPC-treated water to wash a few times, then treat with 50 mM NaOH for 45 minutes. Treat with 20×SSC for 45 minutes. Soak nylon membrane in deionized water, then treat with 20×SSC for 45 minutes. Spread a layer of filter paper on gel support, and soak in 20×SSC. Remove foam. Flip gel over and place on support, and on top of that a thin plastic sheet with a hole dug in the middle. Carefully place the membrane on the gel, remove foam, and spread two sheets of filter paper soaked in 20×SSC on top of membrane. Remove foam. On filter paper spread 10 cm of water absorbent paper and press with a 500 g heavy object. Transfer membrane for 16 hours, changing absorbent paper 2-3 times. Carefully extract the membrane, photograph it and soak in 6×SSC for 5 minutes. For ultraviolet cross-linking, bake at 80° C. for 1 hour. Store at 4° C.

3.2.4 Preparation of Hybridization Template

Upstream primer: 5′-CC GAATTC ATGCACCGGCTCATCTTTGTC-3′. Text in italics refers to protected base, text in bold refers to enzyme digestion site. Underlined text is complementary to Fwa267 nucleic acid sequence 107-127. Downstream primer: 5′-GC CTCGAG TCTTATCGAGGTGGTCTTGAGCTG-3′. Text in italics refers to protected base, text in bold refers to XhoI enzyme digestion site. Underlined text is complementary to Fwa267 nucleic acid sequence 1198-1221.

PCR reaction system: 10 multiple buffer 5.0 ul, deoxyribonucleic acid 2.0 ul. Upstream, downstream primers 3.0 ul each, Taq enzyme 1.0 ul. Fwa267 sequencing plasmid (template) 2.0 ul, sterile water 34 ul. PCR parameters: 94° C. predenaturing for 3 minutes. Then, 94° C. for 3 minutes, 94° C. for 20 seconds, 60° C. for 30 seconds, 72° C. for 80 seconds, 30 cycles. 72° C. for 7 minutes. Purify PCR product with ammonium acetate/alcohol (1:5). Dissolve with 50 ul TE. Determine content volume with agarose, and dilute to 25 ng/μl.

3.2.5 Hybridization

Marker probes: Add PCR product 25 ng (Heat to 98° C. for 4 minutes to denature, cool on ice for 2 minutes) into 0.5 ml centrifuge tube. 5×marker buffer 10 ul, dNTP (without dCTP) 2.0 ul, BSA 2.0 ul, Klenow enzyme 1.0 ul, [α-32P] dCTP 5.0 ul. Add non-nuclease water to bring total volume to 50 ul. Reaction at room temperature 1 to 3 hours.

Prehybridization: Soak membrane in 6×SSC for 5 minutes and adhere to wall of hybridization tube. Remove foam. Add 6 ml Clontech hybridization solution, 68° C. for one hour.

Hybridization: Pour out hybridization fluid, add pre-heated hybridization fluid 6 ml, add probe (denatured at 98° C. for 4 minutes, cooled on ice for 2 minutes). 68° C. for 3 hours.

Wash membrane with 200 ml wash solution I (2×SSC, 0.05% SDS) 4 times, 10 minutes each time. Wash with 200 wash solution II (0.1×SSC, 0.1% SDS) at 50° C. for 20 minutes, then wash at 56° C. for 20 minutes.

Preform: Use filter paper to absorb liquid, wrap in preservative film, and adhere to a sheet of filter paper the same size as x-ray film, press. −70° C. exposure for suitable period of time. Wash preform.

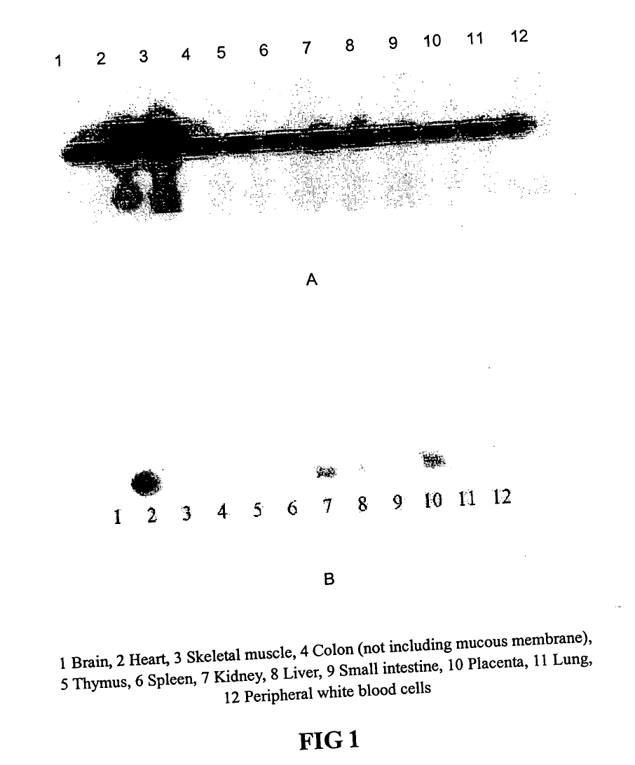

Results: As shown in FIG. 1: β-actin is expressed consistently in many types of tissue (FIG. 1A). Fwa267 selectively expressed at high level in heart. Expression in placenta and kidney cells as well (FIG. 1B).

### Embodiment 4

### Expression of Fwa267 Gene in Tumor Cell Strain

4.1 Test Material

mRNA hybridization membrane containing 8 types of tumor cell lines available from Clontech of the U.S.A. It is used to detect Fwa267 gene distribution in different tumor cell lines. β-actin is used for comparison in this test.

4.2 Northern Hybridization

Refer to Embodiment 3.2

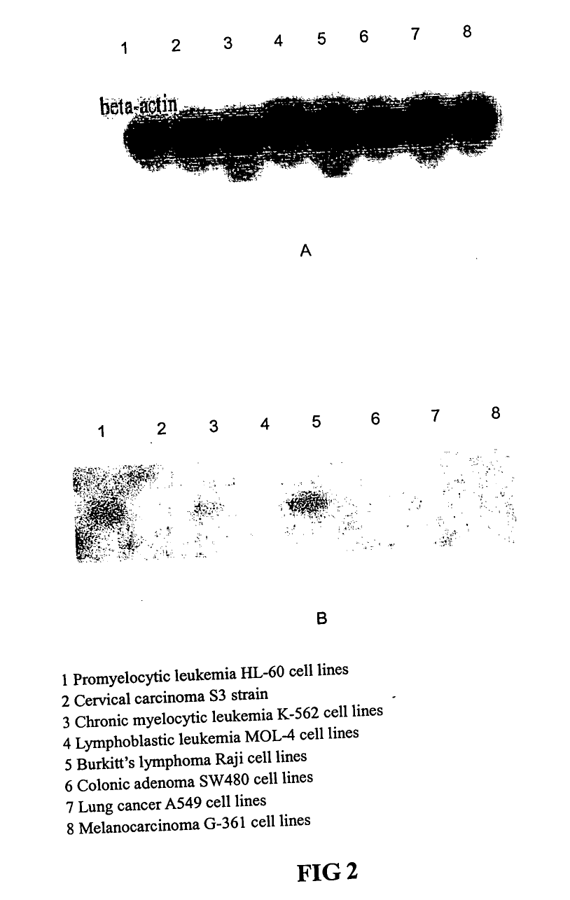

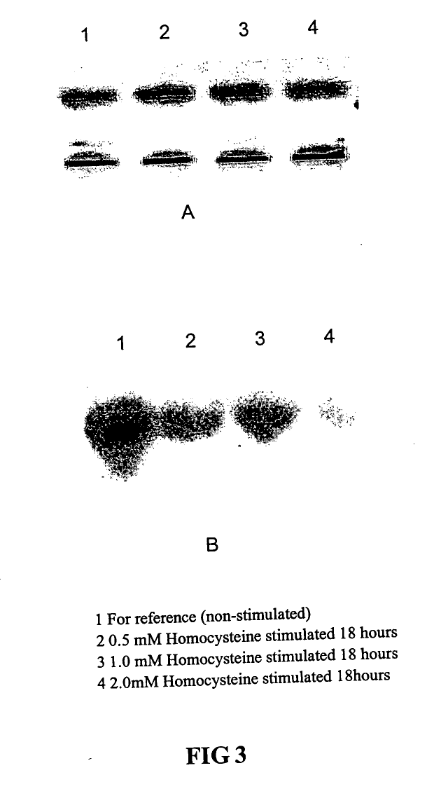

Results: As shown in FIG. 2: β-actin is expressed consistently in several types of tissue (FIG. 2A). Fwa276 is barely expressed in some tumor cell lines, and is expressed at low levels only in promyelocytic leukemia HL-60 cell lines, lymphoblastic leukemia MOL-4 cell lines and Burkitt's lymphoma Raji cell lines (FIG. 2B).

Based upon the results above, it is inferred that Fwa267 is a differentiation factor. Regular adult myocytes are terminally differentiated cells without the ability to proliferate, while tumor cells reproduce uncontrollably. The difference between the proliferative abilities of the two types of cells may be due to the following: the Fwa267 gene in normal adult myocytes is expressed at a high rate, so that it maintains a state of a high rate of differentiation. In tumor cells, however, it is expressed at a low rate, leading to uncontrolled proliferation.

### Embodiment 5

### Different Expressions of Fwa267 Gene in Various Myocytes

5.1 Test Material

Myocytes from fetuses, adults and patients with Tetralogy of Fallot.

5.2 Northern Hybridization

Refer to Embodiment 3.2

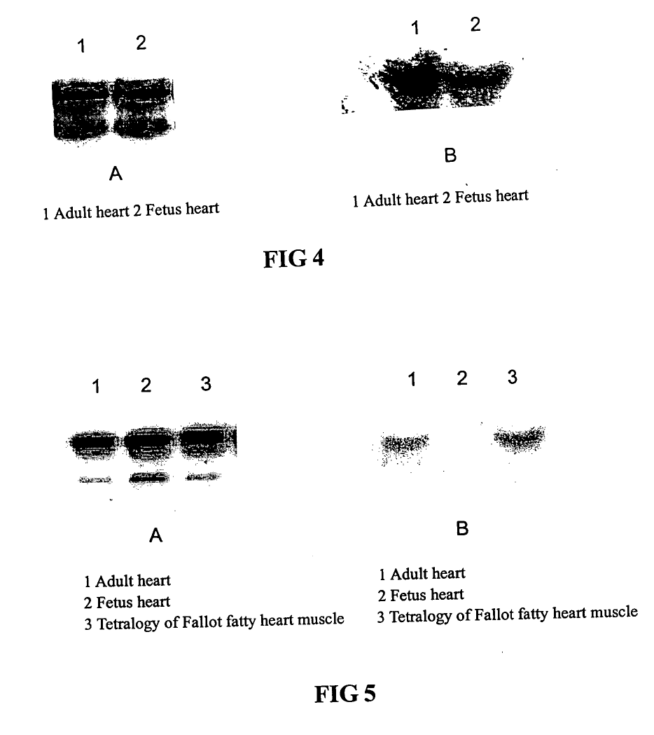

Results: As shown in FIG. 4, Fwa267 gene is not expressed in a fetus heart. While normal adult myocytes are terminally differentiated cells without the ability to proliferate, fetus myocytes still have the ability to proliferate and differentiate. Results reveal that Fwa267 is related to maintaining terminal differentiation of the heart.

As shown in FIG. 5, expression of the Fwa267 gene in Tetralogy of Fallot's fatty heart muscle and in adult hearts is normal, but both are higher than in fetus hearts. Tetralogy of Fallot heart muscle cells undergo necrotic fibrosis, but Fwa276's expression reveals that it is related to the cell's differentiation.

### Embodiment 6

### Effect of Homocysteine Upon Smooth Muscle Cell's Expression of Fwa267

Homocysteine has recently been proven to be an independent factor in the cause of arteriosclerosis, cerebral strokes, coronary heart disease, myocardial infarction and peripheral artery disease. It stimulates cells, especially the reproduction of smooth muscle cells. The present test uses different concentrations of homocysteine to treat smooth muscle cells of the human aorta cultivated outside of the body, to observe variations in the expression of Fwa267 in the cells.

6.1 Resuscitation of Continuous Cells

Cryopreserved smooth muscle primary cells from a human aorta are quickly extracted out of liquid nitrogen and put into a 37° C. water bath. After they have completely thawed, cell suspension is inoculated in a 25 cm culture flask in which 5 ml RPMI or DMEM containing 10% FBS is already present. At 37° C., with a CO₂ concentration of 5%, cultivate in incubator overnight. Change liquid the next day. The cultivating liquid is still RPMI or DMEM containing 10% FBS.

6.2 Continuous Cultivation

Once cells have grown and basically fused (coverage rate of about 80%), discard original cultivating liquid. Use 1×PBS (pH 7.4) to rinse the surface of the cells twice, then add 0.125% trypsin and digest at 37° C. for 5 to 10 minutes. Observe under a microscope. After cells retract and become round, with some of them floating, add a little cultivating liquid containing 10% FBS to stop digestion, then use a straw to blow repeatedly on the surface of the cells. Collect the digested liquid and centrifuge at 1,000 RPM for 30 seconds. Discard supernatant. Add cultivating liquid containing 10% FBS, blow on it, mix evenly, and extract 0.1 ml using 1×PBS to dilute to 1.0 ml. Count. For each 100,000 cells per ml inoculate cell suspension into the culture flask which already has 5 ml RPMI or DMEM containing 10% FBS. At 37° C., with a CO₂ concentration of 5%, cultivate in an incubator. Continue 3 times using the same method until the desired amount is reached. Fused cells (coverage rate of about 80%) are [sic], discard original cultivating liquid, and replace it with cultivating liquid containing 0.4% FBS and continue cultivating for a period of 24 to 72 hours so that cell growth reaches the quiescent stage.

6.3 Homocysteine Stimulation of Human Smooth Muscle Cells

To prepare 150 mM homocysteine, filter through a 0.22 um filter and use DMEM to dilute to 7.5 mM, 15 mM and 30 mM concentrations. Stimulate cells with 0.5 mM, 1.0 mM and 2.0 mM concentrations of homocysteine for 18 hours. For the 1.0 mM stage add 100 uM soy genistein and cultivate for 6 hours. Retrieve the supernatant. Guanidinium isothiocyanate (GTC) is used to harvest cells.

6.4 Northern Hybridization

Use three flasks of the cells mentioned above. Extract mRNA. Refer to Embodiment 3.2 for Northern hybridization.

Results: By treating human smooth muscle cells with homocysteine for 18 hours, expression of Fwa267 gene can be inhibited. Inhibition is increased with higher concentrations of homocysteine.

The results reveal that cells with homocysteine with increased reproduction may inhibit expression of the Fwa267 gene, which removes Fwa267's inhibition of cell differentiation.

### Embodiment 7

### Expression of Fwa267 Gene in Hearts that are Normal, Those with Chronic Heart Failure and Those with Subacute Heart Failure

7.1 Establishing a Chronic Heart Failure Model

Fifty male Sprague-Dawley mice (250-300 g each) were purchased from the animal room of this institution. Standard samples were provided by the Beijing Animal Center. Drinking water was tap water. Mice took water and food at will.

Weight measurement was based upon body weight. Ketamine and valium were injected into the abdominal cavity to anesthetize the mice. A tube was inserted into the trachea and a breathing apparatus was connected. A significant ST rise on the cardio monitor was a sign of successful ligation. Upon regaining consciousness the breathing apparatus was disconnected. Feeding conditions remained the same as before the operation. The model was complete in 50 days.

7.2 Establishing a Subacute Heart Failure Model

10 male Wistar mice (250 g each) were purchased from PLA Hospital 301. Feeding conditions were the same as in the previous model.

Noradrenaline was injected into the abdominal cavity at 30 mg per day for 4 days in succession. On the fifth day the model was ready for use.

7.3 RNA-RNA in situ Hybridization

In situ hybridization is based upon the principle of base complementation, using probes to hybridize with specified mRna or DNA within the cell to reveal genes and expression products. Said reaction is extremely sensitive, and is able to detect momentary gene expressions during tissue differentiation. Hybridization reagent DIG RNA Labeling kit (Cat. No. 1175125) is available from German company Beohringer Mannheim.

7.3.1 Preparation of Probes

Using special primer PCR, 200-300 bg Fwa267 nucleic acid fragment (222-504 nucleotide) clones enter the T Vector. Recombinant plasmid is amplified and purified. T7 and SP6 primers are used for amplification of PCR. Determine direction of gene insertion, and sequence (mRNA and AntimRNA). Add T7 RNA polymerase synthetic antisense probes and SP6 RNA polymerase plus-sense probes (to test the system for dependability).

7.3.2 Probe Marker

Use phenol/chloroform to extract transcribed linear DNA, then use alcohol for precipitation. A centrifuge tube without any RNA enzymes is placed on ice. Add 1 μl purified linear DNA, 2 μl NTP marker mixed solution. 2 μl 10×transcription buffer, 1 μl RNA enzyme inhibitor, 2 μl (40 u) SP6 or T7 TNA polymerase, for a total of 20 μl. Mix evenly, centrifuge lightly. Add 20 u DNA enzyme I not containing RNA, and heat to 37° C. for 15 minutes. Add 2 μl 0e2 mol/L EDTA (pH 8.0) to terminate the reaction.

Then, add 2.5 μl 4 mol/L Licl, and 75 μl alcohol (−20° C.) and mix evenly. Let sit at −70° C. for at least 30 minutes (or −20° C. for at least 2 hours). Centrifuge at 12,000 rpm, wash with 70% cold ethanol, and dry. Add 100 μl DEPC water and 20 u RNase. Let sit at 37° C. for 30 minutes. Separate and store at −20° C.

7.3.3 Preparation of Glass Slides and Samples

Thoroughly clean slides and sterilize at high pressure for 30 minutes. Soak for a few moments in a wash containing polylysine (0.01%), then dry. Keep at 4° C.

Rinse a fresh sample (from mouse heart and aorta) at 0.4×0.4 cm with 1×PBS twice, and place on the solid head of a frozen microtome. Slice to 8-10 μm and place on a glass slide containing 1 mg/ml polylysine to dry. Use 4% paraformaldehyde to solidify for 15-20 minutes. Wash twice with 1×PBS, 5 minutes each time.

7.3.4 Pre-hybridization Treatment

Soak in 0.2 N HCl for 25 minutes, then soak in 0.3% Triton X-100 for 5 minutes. Wash twice with 1×PBS, 5 minutes each time. Use 4% paraformaldehyde to solidify for 6 minutes. Wash twice with 1×PBS, 5 minutes each time. Add 5 ml acetic anhydride into 1,000 ml 0.1 mol/L triethanolamine (pH 8.0) solution. After it is completely dissolved, let sit for 10 minutes. The whole reaction takes place at room temperature.

7.3.5 Hybridization Reaction

The hybridization solution contains 5 ml deionized formamide, 2.5 ml 20×SSC, 500 μl 100×Denhardt's solution (10 g ficoll, 10 g polyvinylpyrrolidone, 10 g bovine serum albumin, 500 ml sterilized twice-distilled water), 500 μl 10% SDS, 100 μl 10 mg/ml denatured salmon sperm DNA and 400 ul DEPC water.

Remove sample from acetylate solution, dry all four ends with filter paper and draw a circle around the sample. Add pre-hybridization solution 30 ul into the circle and incubate in wet box at 42° C. for 2 hours. Throw out pre-hybridization solution, and cover the Parafilm membrane with 25 ul of hybridization solution. Cultivate in a sealed wet box at 42° C. for 16 hours.

7.3.6 Post-Hybridization Treatment

Extract glass slide from wet box and remove the Parafilm membrane. Rinse three times at room temperature with 2×SSC, 10 minutes each time. Rinse three times at room temperature with 1×SSC, 10 minutes each time. Rinse twice at 50° C. with 0.1×SSC, 15 minutes each time.

If the base is too high, place glass slide in a solution containing 20 μg/ml RNA (0.5 mol/L NaCl, 10 mmol/L Cris-Cl, pH 8.0) and digest at 37° C. for 30 minutes. Rinse with said solution without RNA enzyme at 37° C. for 30 minutes. Wash the membrane.

7.3.7 Color Reaction

Soak glass slide in a wash solution (1 M maleic acid, 0.15 M NaCl, 0.3% (V/V) Tween-20). Incubate for 30 minutes in a blocking solution (blocking solution included in reagent kit at 10% (W/V), add maleic acid buffer (0.1 mol/L maleic acid, 0.15 mol/L NaCl), dissolve to 1:10 for use). Add 20 ml antibody solution, incubate for 30 minutes. Wash twice with 100 ml wash solution, 15 minutes each time. Add 20 ml tracer solution (0.1 M Tris-HCl, 0.1 M NaCl, pH 9.5), balance 2-5 minutes. Cultivate in 10 ml of freshly-prepared color solution (add 200 ul NBT/BCIP to 10 ml of tracer solution). Avoid light, do not agitate. Rinse with sterilized twice-distilled water or TE.

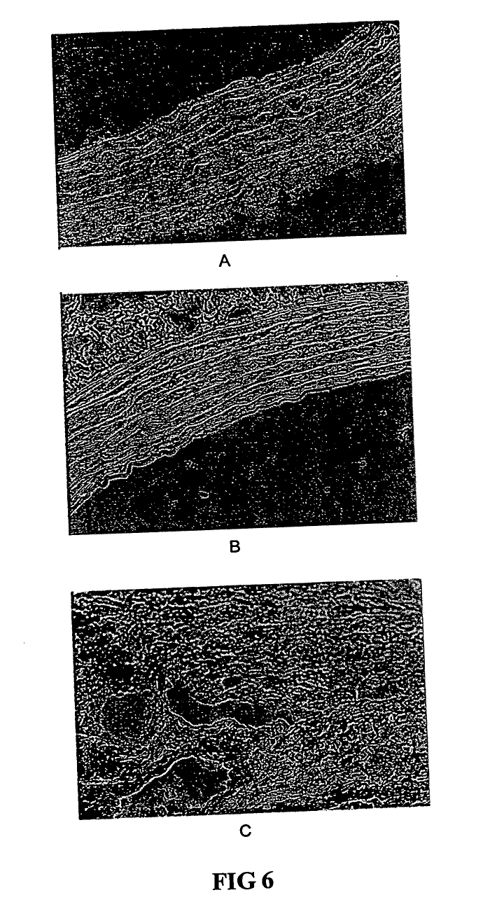

Results: As shown in FIG. 6, the expression of Fwa267 in normal aorta is comparatively lower, while the expression in animal aorta with subacute heart failure or chronic heart failure is significantly higher.

### Embodiment 8

### Immunohistochemical Analysis

8.1 Test Material

Human acute myocardial infarction complicated by ventricular wall tumor tissue

8.2 Immunohistochemical Analysis

Use correctly recombinant prokaryotic expression plasmid to transfect E. coli B1 21 competent cells. Break up cells by ultrasound. Put through 10% denatured polyacrylamide electrophoresis to determine the targeted gene band. Apply ultrasound and electroelution. Obtain fused protein from the inclusion body, and determine targeted protein by Western blotting. The serum obtained by immunizing the animal is the primary antibody used in this test.

Attach paraffin section 5 um to APES glass slide containing polylysine and bake at 75° C. for 2 hours. After removing paraffin from xylene and putting through gradient alcohol, place the section into water, 3% H₂O₂ for 10 minutes. Wash three times with distilled water, then place in EDTA antigen solution (pH 8.0) and bake in microwave (96-98° C.) for 10 minutes. Cool at room temperature 20-30 minutes and wash three times with distilled water. Place in 1×PBS buffer for 5 minutes, then into 10% normal rabbit serum for 20 minutes. Add an appropriately diluted primary antibody. Incubate overnight at 4° C. Wash three times with 1×PBS, add biotin labeled sheep anti-rabbit secondary antibody and let sit at room temperature for 10 minutes. Wash three times with 1×PBS, add enzyme linked streptavidin tertiary antibody and let sit at room temperature for 10 minutes. Wash three times with 1×PBS, then perform DAB color development. Wash with distilled water three times, stain the cell nuclei with hematoxylin, remove water and seal the slide.

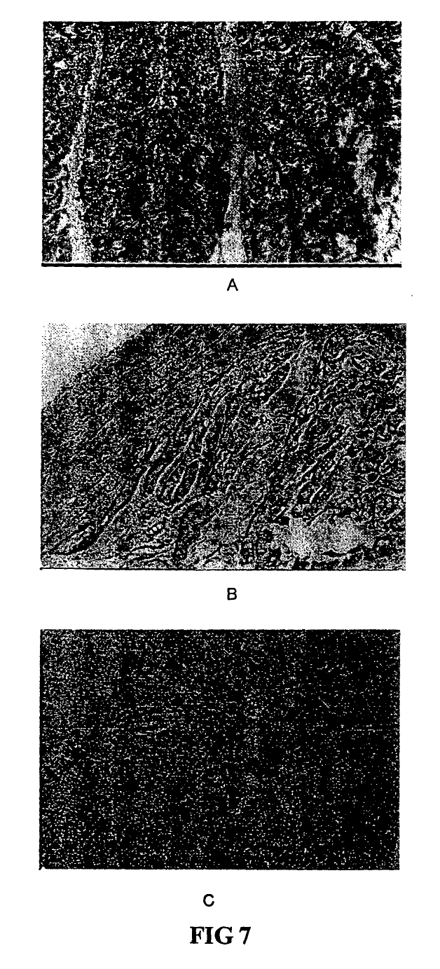

Results: As shown in FIG. 7, normal myocytes express a low level of Fwa267, but ventricular wall tumor tissue expresses a high level of Fwa267.

### Embodiment 9

### Fwa267 Gene Recombination Outside the Body and Protein Expression

9.1 Constructing Recombinant Vectors

9.1.1 Enzyme Digestion

The vector is pGEX-5X-1. Take 2.0 ul pGEX-5X-1 vector, 5.0 ul buffer, 2.0 ul XhoI, 2.0 ul EcoRI, 39 ul sterilized water, and allow to digest at 37° C. for 16 hours. Purify the digested product.

PCR primer (upstream 5′-CC GAATTC ATGCACCGGCTCATCTTTGTC-3′, downstream 5′-GC CTCGAG TCTTATCGAGGTGGTCTTGAGCTG-3′) is the same as Northern hybridization. The PCR reaction is the same as in Embodiment 3.2.4. Take 10 ul PCR purified product, 5.0 ul buffer, 2.0 ul XhoI, 2.0 ul EcoRI, 31 ul sterilized water, and allow to digest at 37° C. for 16 hours. Purify the digested product.

9.1.2 Linking

2.0 ul pGEX-5X-1 digestion product, 2.0 ul PCR digestion product, 2.0 ul 10×buffer, 1.0 ul Tp ligase and 13 ul sterilized water are linked at 16° C. for 6 hours.

9.1.3 Transformation

Take 1.0 ul of the linked product above and add 200 ul E. coli DH5 α competent cells. Mix evenly. Place on ice for 30 minutes. Heat at 42° C. for 60 seconds, and then place back on ice for 2 minutes. Add 800 ul SOC culture medium and agitate at 37° C. (less than 225 rpm) for 45 minutes. Extract 100 ul and spread it on an ampicillin-resistant LB base, at 37° C. for 30 minutes, to completely absorb the liquid. Incubate at 37° C. for 16 hours. Place single clones on 5 ml LB 50 ug Amp culture base at 37° C. and shake for 16 hours.

9.1.4 Extracting Plasmid

Centrifuge bacterial solution at 2,000 rpm for 10 minutes, and discard the supernatant. In the precipitant add pre-cooled solution I (25 mM Tris-HCl, pH 8.0, 2.0 mM EDTA, 50 mM glucose) 140 ul to suspend bacteria. Cleave bacteria with 280 ul freshly-prepared solution II (0.2 M NaOH, 1% SDS). Add 450 ul pre-cooled solution III (4.0 M potassium acetate) and mix evenly. Centrifuge at 4,000 rpm for 10 minutes, and then at 12,000 rpm for 10 minutes. Extract supernatant. Add 0.6 of volume isopropanol, and centrifuge as above. Dissolve and precipitate with 100 ul TE (10 mM Tris-HCl, 0.1 mM EDTA, pH 8.0) and 20 ug/ml RNA enzyme at 37° C. for 30 minutes. Add 600 ul ammonium acetate/alcohol (1:5) and let sit at −70° C. for 30 minutes. After centrifuging, wash with 75% alcohol. Use TE 100 ul dissolved plasmid to assay OD260/280 content.

9.1.5 Selection of Positive Clones

Use the PCR method to identify positive clones.

9.2 Expression in Prokaryotic System

Transform E. coli BL21 competent cells using recombinant plasmid. Transform and incubate bacteria. Add upper bacterial solution in 1:100 proportion to 50 ml 2×YTA culture medium. Agitate at 37° C. for 4-6 hours so that OD600 is 0.6-0.8. Add IPTG, render concentration to 0.4 mM, and continue agitation for 4 hours. Centrifuge at 7,700 rpm for 10 minutes. Discard supernatant. Suspend bacteria with ice pre-cooled 1×PBS (50 ul/ml bacterial solution). Break up bacteria by ultrasound. Centrifuge at 10,000 rpm for 10 minutes. Add sample buffer to supernatant and precipitant, and denature at 95-100° C. for 5 minutes. Undergo electrophoresis for 10% denaturing polyacrylamide gel, and wait for bromphenol blue to sink to the bottom. Dye, destain, and confirm target band.

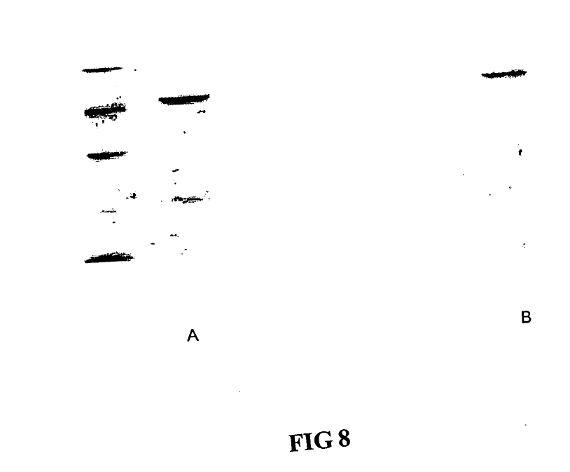

Results: As shown in FIG. 8A, with the low molecular weight marker from Shanghai Lizhu Dongfeng Biotechnology Co., Ltd. used as reference, the Fwa267 protein's molecular weight is about 40 KD.

9.3 Isolating the Inclusion Body

Add bacterial solution and lysate at a proportional volume of 5:1, respectively, to the lysis buffer (1 M PMSF, 1 mg/ml lysozyme dissolved in PBS) and bathe in ice water for 20 minutes. Add Triton-X100 with a final concentration of 1%, and bathe in ice water for 10 minutes. Apply 20 Hz ultrasound for 30 seconds, then centrifuge at 15,000 rpm at 4° C. for 25 minutes. Retain a small amount of supernatant for future use. Add 5 times volume inclusion body wash into the precipitant and re-suspend the inclusion body. Apply 20 Hz ultrasound for 30 seconds, then centrifuge at 15,000 rpm at 4° C. for 25 minutes. Repeat the process 4-5 times. Centrifuge at 3,000 rpm for 10 minutes, and discard supernatant. Centrifuge at 15,000 rpm for 15 minutes, and re-suspend and precipitate in denatured buffer I (2 mM DTT, 2 mM EDTA pH 8.0) (10 ml/g), and apply ultrasound for 30 seconds. Re-suspend and precipitate in denatured buffer II(40 mM NaOH (10 ml/g)) and apply ultrasound for 1 minute. Re-suspend and precipitate in ¼ volume denatured buffer III (40% glycerol, 50 mM Tris-Cl, pH 8.0, 1 ul PMSF) and bathe in ice water for 5 minutes. Centrifuge at 15,000 rpm at 4° C. for 1 hour. Collect supernatant. Store at −20° C.

9.4 Western Blotting Hybridization

Use electrophoresis to isolate the bulky product. Cut out the target band and place in a dialysis bag. Perform electrophoresis at 50 V for 12-16 hours, then 100 V for 2 hours. Reverse polarity for 1 minute. Discard gel. Apply PEG concentrate, 1×PBS dialysis for 16 hours. Change solution once at 4 hours.

Take purified protein 20 ul, add 5 ul 5×sample buffer, perform electrophoresis at 100 V for 2 hours on 12% SDS-PAGE gel. Remove gel, place gel foam in solution for 30 minutes. Cut 6 filter papers to the same size as the membrane, and 1 cellulose nitrate membrane. Place gel on the negative electrode and the cellulose nitrate membrane on the positive electrode. Add three sheets of filter paper on each end and place on semi-dry membrane transfer unit. Transfer at 11 mA for 1 hour (0.65 mA/cm²). Retrieve the membrane and place in 10 ml confining solution at room temperature for 1 hour. Pour out solution, then add 20 ml TBST to wash membrane for 5 minutes, then repeat. At a proportion of 1:20,000 dilute primary antibody, add 1 ul primary antibody and 20 ml confining solution, and let sit for 1 hour at room temperature. Pour out the confining solution and wash the membrane. At a proportion of 1:1,000 dilute secondary antibody, add 10 ul rabbit anti-sheep I gG/AP+10 ml confining solution, then let sit for 1 hour at room temperature. Pour out the confining solution and wash the membrane. Add 20 ml PBS and wash the membrane for 5 minutes, then repeat. Add 20 ml alkaline phosphatase buffer and wash the membrane for 5 minutes. To 33 ul NBT add 5 ml alkaline phosphatase buffer 16.5 ul BCIP, and develop color at room temperature for 5 minutes. Stop color development. Add 20 ml high pressure water and wash for 5 minutes.

Results: As shown in FIG. 8B, the hybridization of the Fwa267 protein band is at approximately 40 KD.

The present invention is not limited to the embodiments above, which are given merely to describe certain aspects of the invention. Functionally equivalent methods and substances are included within the scope of the present invention. Based upon the descriptions and drawings given herein, variations to this invention will be easily ascertained by technical personnel in this field. Such variations are contained within the scope of the claims of the present invention.

1. An isolated polynucleotide selected from the group consisting of:
(a) a polynucleotide having a sequence encoded as the polypeptide of SEQ ID NO:2;
(b) a polynucleotide that is a variant of naturally occurring (a); and
(c) a polynucleotide able to hybridize with (a) and having at least 85% homology with (a).

2. A polynucleotide of claim 1, wherein said polynucleotide is DNA.

3. A polynucleotide of claim 1, wherein said polynucleotide is RNA.

4. A polynucleotide of claim 1, wherein said polynucleotide is genome DNA.

5. A polynucleotide of claim 1, wherein said polynucleotide has the sequence set forth in SEQ ID NO: 1.

6. A polynucleotide of claim 1, wherein said polynucleotide has the sequence set forth as nucleotides 107 to 1219 of SEQ ID NO:1.

7. A polynucleotide of claim 2, wherein said nucleotide has a sequence encoded as the polypeptide of SEQ ID NO:2.

8. A vector comprising the DNA of claim 2.

9. A host cell transformed or transfected by the vector of claim 8.

10. A method for producing polypeptide which includes said DNA-encoded polypeptide expressed in the host cell of claim 9.

11. A polypeptide selected from the group consisting of:
(a) a polypeptide having an inferred amino acid sequence set forth in SEQ ID NO:2;
(b) a polypeptide which is an active fragment, analog or derivative of (a); and
(c) a polypeptide having at least 85% homology with the amino acid sequence set forth in SEQ ID NO:2.

12. An antibody of the polypeptide of claim 11.

13. A compound which inhibits the activation of the polypeptide of claim 11.

14. A pharmaceutical composition containing an effective amount of the polypeptide of claim 11 or its active fragment, and one or more pharmaceutically acceptable vectors or excipients.

15. A use for the polypeptide of claim 11 in the preparation of pharmaceutical compositions used to treat cardio-cerebral-vascular diseases.

16. A method for treating patients requiring Fwa267, comprising administering a therapeutically effective dosage of the polypeptide of claim 11 to a patient.

17. A method of claim 16, wherein said patient is provided DNA encoding said polypeptide, and said polypeptide is expressed within the body of said patient.

18. A method for diagnosing disease or susceptibility to disease comprising assaying for a mutation of the polynucleotide of claim 1.

19. A method for diagnosing disease, comprising analyzing whether or not a polypeptide of claim 11 exists in a sample from a host cell.
